(19) United States
(12) Patent Application Publication
(10) Pub. No.: US 2006/0013898 A1
(43) Pub. Date: Jan. 19, 2006

(54) Aromatherapy system

(76) Inventor: David Mobley, Ft. Worth, TX (US)

Correspondence Address: WELSH & FLAXMAN LLC, 2000 DUKE STREET, SUITE 100, ALEXANDRIA, VA, 22314, US

(21) Appl. No.: 10/891,095

(22) Filed: Jul. 15, 2004

(51) Int. Cl. A61K 36/18; A61F 13/00; A61K 9/70

(52) U.S. Cl. 424/725; 424/443

(57) ABSTRACT

An aromatherapy system includes a fragrance impregnated card and a vial shaped and dimensioned for receiving the fragrance impregnated card. The vial includes a selectively removable closure limiting airflow within the vial for maintaining fragrance within the fragrance impregnated card. The fragrance impregnated card has a rolled configuration focusing fragrance within a center of the vial and positioning the fragrance impregnated card within the vial.

### BACKGROUND OF THE INVENTION

1. Field of the Invention

The invention relates to an aromatherapy delivery system. More particularly, the invention relates to an aromatherapy delivery system employing a coiled, scented substrate placed within a package for delivery of the fragrance impregnated within the substrate.

2. Description of the Prior Art

It is well known that humans encounter positive sensory feedback when exposed to specific fragrances and scents. The beneficial affects, whether they are medicinal or emotional, are evidenced by the many dollars spent by consumers of fragrances and scent emitting devices. In fact, it is known that consumers spend billions of dollars per year purchasing perfumes, colognes, air fresheners and other scented products that emit a desirable and pleasing fragrance. In addition to fragrances providing a merely desirable aroma, recent developments in holistic medicine suggest that aromatherapy may produce beneficial psychological responses. For example, it is contemplated aromatherapy may be utilized in controlling weight loss, smoking, stress reduction, etc.

With the foregoing in mind, mechanisms for delivering scents and aromas to consumers in a convenient and inexpensive manner are needed. Many early mechanisms for fragrance dispensing rely upon applying the fragrances directly to the skin of consumers. However, many consumers are allergic to direct application of the fragrances to their skin. As such, devices employing various dispensing techniques have been developed for dispersing fragrances through the air and toward the olfactory system of a human being. However, current dispensing techniques are often expensive and cumbersome to utilize. As such, a need continues to exist for an efficient, convenient and inexpensive dispensing system for providing consumers with desirable fragrances and scents. The present invention provides such a dispensing system.

### SUMMARY OF THE INVENTION

It is, therefore, an object of the present invention to provide an aromatherapy system including a fragrance impregnated card and a vial shaped and dimensioned for receiving the fragrance impregnated card. The vial includes a selectively removable closure limiting airflow within the vial for maintaining fragrance within the fragrance impregnated card. The fragrance impregnated card has a rolled configuration focusing fragrance within a center of the vial and positioning the fragrance impregnated card within the vial.

It is also an object of the present invention to provide an aromatherapy system including a roller for selectively rolling the fragrance impregnated card.

It is another object of the present invention to provide an aromatherapy system wherein the roller includes a base and a rotating arm.

It is a further object of the present invention to provide an aromatherapy system wherein the rotating arm includes a central slot shaped and dimensioned for receipt of the fragrance impregnated card.

It is also another object of the present invention to provide an aromatherapy system wherein the vial is made of plastic.

It is still another object of the present invention to provide an aromatherapy system wherein the vial is made of polypropylene.

It is yet a further object of the present invention to provide an aromatherapy system wherein the card is composed of approximately 95 lb to 105 lb blotter card stock.

It is stilt another object of the present invention to provide an aromatherapy system wherein the fragrance is composed of natural aromatics.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a perspective view of a fragrance impregnated card in accordance with the present invention.

FIG. 2 is a perspective view of a rolled fragrance impregnated card.

FIG. 3 is a side view of a vial.

FIG. 4 is a cross sectional view of the vial with the fragrance impregnated card therein.

FIG. 5 is a side view of the roller.

### DESCRIPTION OF THE PREFERRED EMBODIMENT

The detailed embodiment of the present invention is disclosed herein. It should be understood, however, that the disclosed embodiment is merely exemplary of the invention, which may be embodied in various forms. Therefore, the details disclosed herein are not to be interpreted as limiting, but merely as the basis for the claims and as a basis for teaching one skilled in the art how to make and/or use the invention.

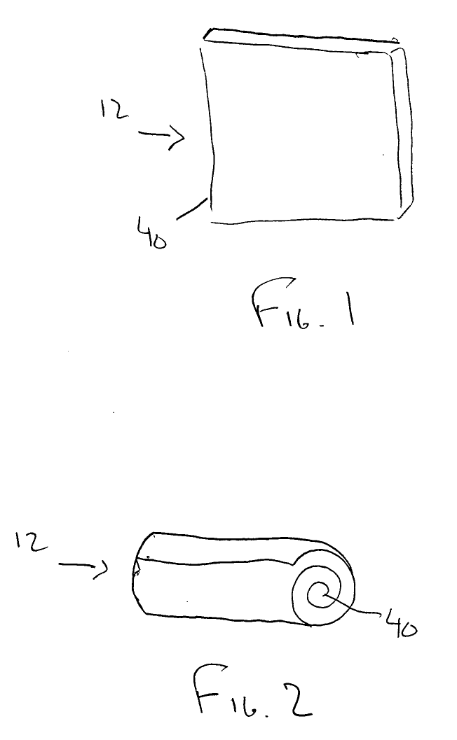

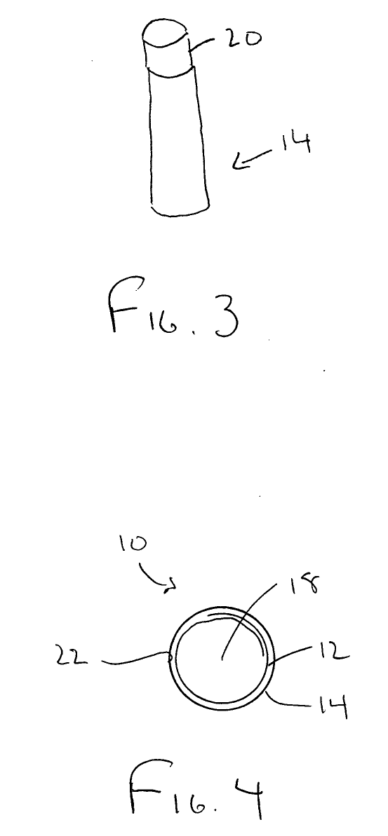

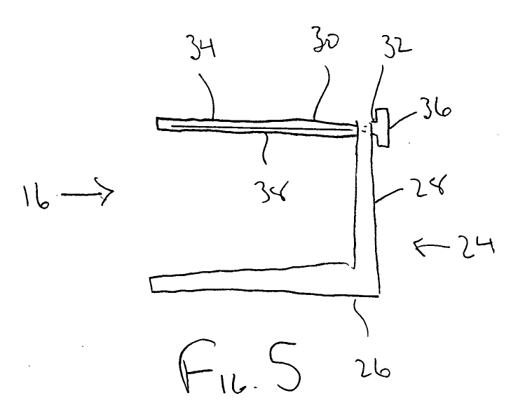

With reference to FIGS. 1, 2, 3, 4 and 5, an aromatherapy system 10 is disclosed. The aromatherapy system 10 of the present invention provides a convenient, portable and cost effective way in which to provide consumers seeking aromatherapy with such a system.

The system includes a fragrance impregnated card 12, a vial 14 and a roller 16. With regard to the fragrance impregnated card 12, it is composed of card stock. In accordance with a preferred embodiment of the present invention, the card stock is approximately 95-105 lb blotter card stock available from a variety of suppliers. The card 12 is impregnated with a blend of natural aromatics in a carrier. As those skilled in the art will appreciate, a variety of aromatics may be employed without departing from the spirit of the present invention.

The fragrance impregnated card 12 is ultimately shaped for placement within the vial 14. In accordance with a preferred embodiment of the present invention, the fragrance impregnated card 12 is rolled prior to placement within the vial 14. In this way, fragrances are focused within a center 18 of the vial 14 for ready access as an individual removes the closure 20. More specifically, and as will be discussed below in even greater detail when the roller 16 itself is discussed, the fragrance impregnated card 12 is rolled in much the same manner one would roll a poster for insertion within a storage tube. Once the fragrance impregnated card 12 is placed within the vial 14, its resilience will bias it toward the inner walls 22 of the vial 14 creating a central space 18 in which the fragrance emanating from the fragrance impregnated card 12 gathers for inhalation by an individual using the present system 10.

The vial 14 is shaped and dimensioned for receiving the fragrance impregnated card 12. The vial 14 includes a selectively removable closure 20 limiting airflow within the vial 14 for maintaining fragrance within the fragrance impregnated card 12. The closure 20 employed in accordance with a preferred embodiment of the present invention, is a screw on cap, although those skilled in the art will appreciate that other closure structures may be employed without departing from the spirit of the present invention.

The vial 14 is preferably made of plastic, particularly, polypropylene. However, other materials may be used without departing from the spirit of the present invention. Any material chosen for use in accordance with the present invention should provide an airtight environment for the fragrance impregnated card 12 and be nonreactive with the fragrance and/or card.

As discussed above, the system also includes roller 16. The roller 16 is designed to conveniently roll fragrance impregnated cards 12 for easy insertion within the vial 14. The need for the roller 16 is dictated by the fact that fragrance impregnated cards 12 will emit all of the impregnated fragrance and require replacement. As such, consumers may purchase replacement fragrance impregnated cards 12. These cards 12 must then be rolled for insertion within the vial 14.

The roller 16 includes a support structure 24 composed of a weighted base 26 and an extension arm 28 extending therefrom. A rotating arm 30 is secured to the extension arm 28. The rotating arm 30 includes a first end 32 on one side of the extension arm 28 and a second end 34 on the opposite side of the extension arm 28. With this in mind, the rotating arm 30 passes through the extension arm 28 and is supported for rotation relative thereto.

The first end 32 of the rotation arm 30 is provided with a knob or handle 36 for actuation by a user. The second end 34 of the rotating arm 30 includes a slit 38 shaped and dimensioned for receiving an edge 40 of the fragrance impregnated card 12.

In use, an edge 40 of the fragrance impregnated card 12 is inserted within the slit 38 and the rotating arm 30 is rotated in either a clockwise or counterclockwise direction. As the rotating arm 30 is rotated, the card 12 is forced to coil around the second end 34 of the rotating arm 30. The rotating arm 30 is shaped and dimensioned to roll the card 12 in a desired diameter. Once the card 12 is fully rolled it may be removed from the slit 38 and slid off the rotating arm 30 and inserted into the vial 14.

While the preferred embodiments have been shown and described, it will be understood that there is no intent to limit the invention by such disclosure, but rather, is intended to cover all modifications and alternate constructions falling within the spirit and scope of the invention as defined in the appended claims.

1. An aromatherapy system, comprising:
a fragrance impregnated card;
a vial shaped and dimensioned for receiving the fragrance impregnated card, the vial including a selectively removable closure limiting airflow within the vial for maintaining fragrance within the fragrance impregnated card;
the fragrance impregnated card having a rolled configuration focusing fragrance within a center of the vial and positioning the fragrance impregnated card within the vial.

2. The aromatherapy system according to claim 1, further including a roller for selectively rolling the fragrance impregnated card.

3. The aromatherapy system according to claim 2, wherein the roller includes a base and a rotating arm.

4. The aromatherapy system according to claim 3, wherein the rotating arm includes a central slot shaped and dimensioned for receipt of the fragrance impregnated card.

5. The aromatherapy system according to claim 1, wherein the vial is made of plastic.

6. The aromatherapy system according to claim 5, wherein the vial is made of polypropylene.

7. The aromatherapy system according to claim 1, wherein the card is composed of approximately 95 lb to 105 lb blotter card stock.

8. The aromatherapy system according to claim 7, wherein the fragrance is composed of natural aromatics.

9. The aromatherapy system according to claim 1, wherein the fragrance is composed of natural aromatics.
